(19) United States
(12) Patent Application Publication
(10) Pub. No.: US 2021/0147810 A1
(43) Pub. Date: May 20, 2021

(54) SINGLE LUNG CELL-DERIVED ORGANOIDS

(71) Applicant: Rutgers, The State University of New Jersey, New Brunswick, NJ (US)

(72) Inventors: Hatem Sabaawy, Neshanic Station, NJ (US); Sharon Pine, Neshanic Station, NJ (US)

(21) Appl. No.: 16/626,059

(22) PCT Filed: Jun. 28, 2018
(86) PCT No.: PCT/US2018/040036

§ 371 Date: Dec. 23, 2019

Related U.S. Application Data
(60) Provisional application No. 62/526,052, filed on Jun. 28, 2017.

(51) Int. Cl. C12N 5/071; G01N 33/50; A01K 67/027

(52) U.S. Cl. CPC C12N 5/0688; G01N 33/5011; G01N 33/5088; A01K 67/0271; C12N 2501/115; C12N 2501/33; C12N 2513/00; C12N 2501/999; A01K 2267/0331; A01K 2227/105; A01K 2207/15; A01K 2207/12; C12N 2501/998

(57) ABSTRACT

The present invention relates to organoids derived from a single cell, such as a lung cancer cell, and methods and compositions relating to the production and use thereof, including cell culture medium for producing organoids and methods of personalized treatment for lung cancer. The invention further provides a humanized mouse including a lung organoid derived from a patient's lung cell.

### CROSS-REFERENCE TO RELATED APPLICATION

This application claims the benefit of U.S. Provisional Application No. 62/526,052 filed Jun. 28, 2017, the disclosure of which is incorporated herein by reference in its entirety.

### BACKGROUND OF THE INVENTION

The inability to propagate primary tissues represents a major hurdle to understanding the mechanisms of regeneration and the balance of differentiated cells versus stem cells in adult organisms. A need exists to better understand primary human pathological disorders such as injury repair and tumor development. For cancer studies, current cancer models do not adequately represent the molecular and cellular diversity of human cancers. Existing human cancer cell lines lack defined and detailed information regarding the clinical presentation of the cancer and have inherent limitations for deciphering the mechanisms of therapy resistance. For injury repair, there is a lack of understanding of the mechanisms of regeneration and shortage of tissue and organs for transplantation. Therefore, novel methods to maintain primary tissues for cancer, new drug discovery approaches to treat cancer and regenerative medicine indications are needed.

Maintaining the balance between normal differentiated cells and progenitor or stem cells is complex. Adult stem cells provide regeneration of different tissues, organs, or neoplastic growth through responding to cues regulating the balance between cell proliferation, cell differentiation, and cell survival, with the later including balanced control of cell apoptosis, necrosis, senescence and autophagy. Epigenetic changes, which are independent of the genetic instructions but heritable at each cell division, can be the driving force towards initiation or progression of diseases. Tissue stem cells are heterogeneous in their ability to proliferate, self-renew, and differentiate and they can reversibly switch between different subtypes under stress conditions. Tissue stem cells house multiple subtypes with propensities towards multi-lineage differentiation. Hematopoietic stem cells (HSCs), for example, can reversibly acquire three proliferative states: a dormant state in which the cells are in the quiescent stage of the cell cycle, a homeostatic state in which the cells are occasionally cycling to maintain tissue differentiation, and an activated state in which the cells are cycling continuously. The growth and regeneration of many adult stem cell pools are tightly controlled by these genetic and/or epigenetic responses to regulatory signals from growth factors and cytokines secreted through niche interactions and stromal feedback signals.

Lung cancer accounts for one-fourth of all cancer deaths in the U.S. Over half of lung adenocarcinomas have defined oncogenic drivers, such as RAS and EGFR mutations, and ALK fusions. Targeting these proteins clinically with specific inhibitors leads to resistance due to selection of mutant clones or redundant pathways. An impediment to improving lung cancer survival has been the inability to find drug sensitivity models that represent lung cancer and allow for identifying resistance to therapy in patient derived cells before therapy implementation.

Generating disease-specific lung epithelial cells from human lung is particularly important because murine models of lung disease often do not reproduce human lung disease. A prime example of the failure of mouse models to mimic human disease is the Cftr knockout mouse that does not display the Cystic Fibrosis disease-associated lung pathology observed in human patients.

### SUMMARY OF THE INVENTION

Organoids can be used to model and generate new therapies for many devastating lung diseases including lung cancer, chronic obstructive lung disease, pulmonary fibrosis, cystic fibrosis, asthma and bronchopulmonary dysplasia.

In one embodiment, the present invention provides a method of making an organoid from a mammalian lung tissue in vitro comprising: isolating cells from a mammalian lung tissue to provide isolated cells; culturing the isolated cells in a differentiation medium for a time sufficient to enrich for stem cells and induce differentiation; and amplifying the cells by culturing in an extracellular matrix in an organoid medium for a time sufficient to produce organoids.

In another embodiment, the invention provides an in vitro lung organoid comprising epithelial cells (e.g., basal and ciliated cells).

In one embodiment, the in vitro lung organoid is derived from a single epithelial cell of a lung tissue.

In another embodiment, the invention provides an in vitro lung organoid derived from primary lung normal tissue, wherein the organoid comprises epithelial cells.

In another embodiment, the invention provides an in vitro lung organoid derived from primary lung cancer tissue, wherein the organoid comprises epithelial cells.

In some embodiments, a lung organoid as described herein is derived in vitro from primary lung tissue from an African American (AA).

In another embodiment, the invention provides a cell culture medium supplemented with fetal bovine serum (FBS).

In another embodiment, the invention provides a cell culture medium supplemented with FBS, Insulin, and basic fibroblast growth factor (bFGF).

In another embodiment, the invention provides a cell culture medium additionally supplemented with epidermal growth factor (EGF), hydrocortisone, Cholera Toxin, Transferrin and Sodium Selenite.

In another embodiment, the present invention provides a kit including a cell culture medium supplemented with FBS, and a cell culture medium supplemented with FBS, Insulin, bFGF, EGF, hydrocortisone, Cholera Toxin, Transferrin and Sodium Selenite.

In another embodiment, the invention provides a method for identifying agents having anticancer activity against lung cancer cells including selecting at least one test agent, contacting a plurality of patient-specific lung organoids derived from the patient's lung cancer cell with the test agent, determining the number of lung organoids in the presence of the test agent and the absence of the test agent, and identifying an agent having anticancer activity if the number or the growth of the organoid cells is less in the presence of the agent than in the absence of the agent. In another embodiment, the method provides a step of treating the patient with the agent identified as having anticancer activity against the patient-specific organoids but not against normal organoids. A method for identifying agents having anticancer activity against lung cancer cells can further include providing a mouse engrafted with lung cancer cells from the patient and containing a tumor formed from the lung cancer cells; administering the identified agent having anticancer activity to the mouse; and determining if the tumor size is reduced in the presence of the identified agent. In another embodiment, a method for identifying agents having anticancer activity against lung cancer cells can further include providing a humanized mouse engrafted with components of a patient's immune system and lung cancer cells from the patient and containing a tumor formed from the lung cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. This and other embodiments can further include providing a humanized mouse engrafted with lung cancer cells from the patient and containing a tumor formed from the lung cancer cells; administering a control agent to the humanized mouse engrafted with lung cancer cells from the patient; and comparing the size of the tumor in the humanized mouse engrafted with lung cancer cells from the patient to the size of the tumor in the mouse in which the identified agent was administered; and determining if the size of the tumor in the mouse in which the identified agent was administered is reduced relative to the size of the tumor in the humanized mouse engrafted with lung cancer cells from the patient.

In another embodiment of a method for identifying agents having anticancer activity against lung cancer cells, the patient is an African American (AA), and the at least one test agent is an inhibitor of JAK/STAT3 activity.

In another embodiment, the method provides a step of treating the patient with the agent identified as having anticancer activity against the patient-specific organoids but not against normal organoids. In one embodiment of this method, the patient is an African American (AA), and the at least one test agent is an inhibitor of JAK/STAT3 activity.

In another embodiment, the present invention provides normal patient-specific lung organoids, and methods of using such organoids for personalized therapies for lung diseases.

In another embodiment, the present invention provides immune humanized mice with implanted patient-specific lung organoids, and methods of using such mice to identify personalized therapies for lung cancer.

In the methods described herein, the organoids exhibit endogenous three-dimensional organ architecture.

### DETAILED DESCRIPTION OF THE INVENTION

In certain embodiments, the present invention provides lung organoids derived in vitro from normal and cancerous tissues, and methods of making and using such organoids, as well as cell culture media and kits. As disclosed in one embodiment herein, certain growth factors in an in vitro environment containing extracellular matrix molecules in a 3-dimensional culture device may be used to make the organoids.

An organoid is a miniature form of a tissue that is generated in vitro and exhibits endogenous three-dimensional organ architecture. See, e.g., Cantrell and Kuo (2015) Genome Medicine 7:32-34. The organoids of the present invention can be used, for example, to: a) determine genomic targets within tumors and prediction of response to therapies in preclinical and clinical trials; b) detect the activity of an anti-cancer agent by examining the number of surviving organoids after treatment; c) detect the activity of a proliferative agent by determining the number of proliferating cells within each organoid and determining gene expression profiling of relevant pathways; d) detect the activity of a regenerative agent by determining the number of regenerating cells within each organoid and determining gene expression profiling of relevant pathways; e) examine the specificity of agents targeting different cell types within organoids; f) determine the effects of chemotherapy and radiation; g) create mouse models by implantation of the organoid in vivo; h) create preclinical models for examining therapy responses and drug discovery both in vitro and in vivo; and i) determine clonally-targeting anti-cancer therapies.

Accordingly, in one embodiment, the invention provides a method of making an organoid from a mammalian lung tissue in vitro including: isolating cells from a mammalian lung tissue to provide isolated cells; culturing the isolated cells in a differentiation medium for a time sufficient to enrich for stem cells and induce differentiation; and amplifying one or more of the cells by culturing in an extracellular matrix in an organoid medium for a time sufficient to produce organoids. One of ordinary skill in the art can determine a time sufficient to induce differentiation by examining morphological changes associated with differentiation. In one preferred embodiment, the time sufficient to induce differentiation is from about seven to about ten days. In another preferred embodiment, the time sufficient to induce differentiation is about 7 days. One of ordinary skill in the art can determine a time sufficient to induce organoid formation by examining morphological changes associated with organoid formation. In one preferred embodiment, the time sufficient to induce organoid formation is from about 20 to about 60 days. In another preferred embodiment, the time sufficient to induce organoid formation is about 28 days. In one embodiment, the isolated cells are epithelial cells. In one embodiment, a single lung epithelial cell is amplified.

In one preferred embodiment, the differentiation medium comprises advanced-Dulbecco's Modified Eagle Medium (ADMEM) and FBS. ADMEM is typically used at 1×. The concentration of FBS present in the differentiation medium may range from about 1% to about 10%. In a further embodiment, the differentiation medium comprises one or both of Penicillin (500-5000 Units/mL) and Streptomycin (50-500 μg/mL). In a most preferred embodiment, the differentiation medium comprises the following concentrations: ADMEM (Life Technologies) (about 1×); FBS (about 5%); Penicillin (about 1000 Units/mL); and Streptomycin (about 100 μg/mL). The differentiation medium may further comprise or be substituted with other supplements, growth factors, antibiotics, vitamins metabolites, and hormones, synthetic or natural with similar properties as known in the art.

In one preferred embodiment, the organoid medium includes ADMEM, FBS, Insulin and bFGF. The concentration of FBS present in the culture medium may range from about (2-10%). The concentration of Insulin present in the culture medium may range from about 1-100 mg/mL (e.g., 1 mg/mL, 5 mg/mL, 10 mg/mL, 15 mg/mL, 20 mg/mL, 49 mg/mL, 50 mg/mL, 51 mg/mL, 100 mg/mL, etc). The concentration of bFGF present in the culture medium may range from about 0.1-100 mg/mL (e.g., 1 mg/mL, 5 mg/mL, 10 mg/mL, 15 mg/mL, 20 mg/mL, etc). In a preferred embodiment, the organoid medium further comprises EGF and hydrocortisone. The concentration of EGF present in the culture medium may range from about 0.1-100 mg/mL (e.g., 1 mg/mL, 5 mg/mL, 10 mg/mL, 15 mg/mL, 20 mg/mL, 25 mg/mL, etc). The concentration of hydrocortisone present in the culture medium may range from about 0.1-10 mM (e.g., 0.1 mM, 0.5 mM, 0.75 mM, 1 mM, 1.5 mM, 2 mM, 5 mM, etc). In a further embodiment, the organoid medium further includes one or more of the following: Cholera Toxin (0.1-100 ng/mL), Transferrin (0.5-25 ng/mL), Sodium Selenite (0.5-25 ng/mL), Penicillin (500-5000 Units/mL), and Streptomycin (50-500 μg/mL). In a most preferred embodiment, the organoid medium includes the following concentrations: ADMEM at 1×, approximately 5% FBS, approximately 50 mg/mL Insulin, approximately 10 mg/mL bFGF, approximately 20 mg/mL EGF, approximately 1 mM hydrocortisone, approximately 10 ng/mL Cholera Toxin, approximately 5.5 ng/mL Transferrin, approximately 7 ng/mL Sodium Selenite, approximately 1000 Units/mL Penicillin, and approximately 100 m/mL Streptomycin. The organoid medium may further include or be substituted with other supplements, growth factors, antibiotics, vitamins metabolites, and hormones, synthetic or natural with similar properties as known in the art.

In certain embodiments, the cells are from human lung tissue, and human primary lung cancer tissue. In certain embodiments, cells that may be used to make an organoid are human lung stem-like cells. Such cells are known in the art and may be identified and isolated using markers, for example, basal cell markers cytokeratin-5 (CK5) cytokeratin-14 (CK14) and p63, bronchioalveolar stem cell markers BMI1, SOX9, EpCAM⁺, CD24^{Low}, CD49f⁺ and CD104⁺, and lung specific cell markers NKx2.1, E-Cadherin, ID2, clara-cell specific protein (CCSP), surfactant protein precursor C (SPTPC), alveolar type I cell markers FOXJ1 and FOXA2 and multiciliated cell marker HopX.

In one embodiment, the cells are positive for at least one marker selected from the group consisting of NKx2.1, CCSP, SPTPC, FOXJ1 and HopX. In another embodiment, the cells are positive for NKx2.1, CCSP, SPTPC, FOXJ1 and HopX. Such cells may be identified and isolated by methods of cell sorting and laser capture microdissection that are known in the art. For example, in one embodiment, the cells may be isolated by RNA sorting using methods known in the art, such as molecular beacons and the SmartFlare™ probe protocol (EMD Millipore).

In one preferred embodiment, the cells are obtained from surgically excised tissues by subjecting the tissues to mechanical dissociation, collagenase treatment, and filtration.

In certain embodiments the method is performed with a commercially available extracellular matrix such as Matrigel™. Other natural and synthetic extracellular matrices are known in the art for culturing cells. In general, an extracellular matrix comprises laminin, entactin, and collagen. In a preferred embodiment the method is performed using a 3-dimensional culture device (chamber) that mimics an in vivo environment for the culturing of the cells, where preferably the extracellular matrix is formed inside a plate that is capable of inducing the proliferation of stem cells under hypoxic conditions. Such 3-dimensional devices are known in the art. An example of such a device is disclosed by Bansal, N., et al. (2014) Prostate 74, 187-200, the disclosure of which is incorporated herein by reference in its entirety. It has been discovered in accordance with the present invention that the use of a 3-dimensional culture device in a method of making organoids has surprising advantages over other formats, as shown in Table 1.

TABLE 1
Advantages and disadvantages of tested formats

| | Consistency of | | |
|---|---|---|---|
| Format | Organoids | Reproducibility | Efficiency |
| In Matrigel ™ | +++ | +++ | ++++ |
| On Matrigel ™ | + | −−− | ++ |
| Hanging Drop plates | −−− | −−− | −−− |
| Non adherent plate | + | −−− | + |

In another aspect, the invention provides a lung organoid. Normal human lung tissue includes alveolar epithelial cell type I (AEC1) and alveolar epithelial cell type II (AEC2) of the alveoli, and secretory, multiciliated and neuroendocrine cells of the bronchi. Secretory cells such as clara cells are marked by synthesis of CCSP and SCGB1. Neuroendocrine cells express calcitonin, while mucus-producing goblet cells express MUC5a and FOXA3. The lung organoids of the present invention resemble the structures of the primary tissue. Upon histological and immunofluorescence analyses, one of skill in the art can determine that the organoids recreate the human AEC1 and AEC2. Lung tissue origin of organoids can be confirmed by detecting the expression of NKx2.1, SOX9, FOXA2, SPTPC and Hopx.

In another aspect, the invention provides a lung organoid derived in vitro from primary lung cancer tissue. Tumor heterogeneity can be efficiently modeled using the methods described to make an organoid, by mapping the diagnostic dominant clone and tumor subclones from each patient biopsy sample, generating organoids derived from each clone and defining the genetic signature of each clone. A lung organoid derived from primary lung cancer tissue will generally maintain expression of lung lineage-specific markers and the functional secretory profile of the original primary tissue. A lung organoid as described herein can be serially propagated, cryofrozen and regenerated and established as a model for cancer drug discovery and precision therapy.

In another aspect, the invention provides a lung organoid derived in vitro from surgically excised tissues of tumors identified to express histopathological tissue specific and tumorigenic markers. Single cells from these tissues may be isolated with non-contact laser capture microdissection and cell sorting or by RNA sorting, for example using SmartFlare™ probes to generate single cell organoids with known expression features.

The organoids described herein exhibit endogenous three-dimensional organ architecture.

In another embodiment, the invention provides a method for identifying agents having anticancer activity against lung cancer cells from a patient(s) including selecting at least one test agent, contacting a plurality of patient-specific lung organoids derived from the patient's lung cancer cell with the test agent, determining the number of lung organoids in the presence of the test agent and the absence of the test agent, and identifying an agent having anticancer activity if the number or growth of the organoids is less in the presence of the agent than in the absence of the agent. In another embodiment, the method provides a step of treating the patient with the agent identified as having anticancer activity against the patient-specific organoids. A method for identifying agents having anticancer activity can further include providing a mouse engrafted with lung cancer cells from the patient and containing a tumor formed from the lung cancer cells; administering the identified agent having anticancer activity to the mouse; and determining if the tumor size is reduced in the presence of the identified agent.

A method for identifying agents having anticancer activity can further include providing a humanized mouse engrafted with components of a patient's immune system and lung cancer cells from the patient and containing a tumor formed from the lung cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered. In this embodiment, the humanized mice with the patient's immune system can be used to compare the effects of the identified agent (e.g., candidate therapeutic) on tumors in the presence or absence of immune cells to examine a potential role for combination with immunotherapy. These methods can further include providing a humanized mouse (an immune-deficient control mouse) engrafted with lung cancer cells from the patient and containing a tumor formed from the lung cancer cells; administering a control agent to the humanized mouse engrafted with lung cancer cells from the patient; and comparing the size of the tumor in the humanized mouse engrafted with lung cancer cells from the patient to the size of the tumor in the mouse in which the identified agent was administered; and determining if the size of the tumor in the mouse in which the identified agent was administered is reduced relative to the size of the tumor in the humanized mouse engrafted with lung cancer cells from the patient. In this method, if the size of the tumor in the mouse in which the identified agent was administered is reduced relative to the size of the tumor in the humanized mouse engrafted with lung cancer cells from the patient, the identified agent can be confirmed as a successful treatment for cancer in the patient.

In another embodiment, the invention provides a method of selecting a personalized treatment for lung cancer in a subject including: selecting at least one form of treatment, contacting a plurality of lung organoids with the form of treatment, wherein the organoids are derived from lung cancer cells from the subject, determining the number of lung organoids in the presence of the treatment and the absence of the treatment, and selecting the treatment if the number or growth of the lung organoids is less in the presence of the treatment than in the absence of the treatment. Various types of therapy can then be examined using the organoids to determine therapy resistance before initiation, to tailor the therapy for each individual patient based on oncogenic driver expression in the organoids, as well as further study induced clonal selection processes that are the frequent causes of relapse. Various forms, combinations, and types of treatment are known in the art, such as radiation, hormone, chemotherapy, biologic, and bisphosphonate therapy. The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. Terms such as “treating” or “treatment” or “to treat” or “alleviating” or “to alleviate” refer to therapeutic measures that cure, slow down, lessen symptoms of, and/or halt progression of a diagnosed pathologic condition.

The foregoing methods may be facilitated by comparing therapeutic effects in organoids derived from cancer cells and normal cells from the same patient. For example, normal organoids and cancer organoids derived from cells of the same patient can be assessed to determine genetic and epigenetic mutations and gene expression profiles that are cancer-specific, thereby allowing the determination of gene-drug associations and optimization of treatment. Such comparisons also allow one to predict a therapeutic response and to personalize treatment in a specific patient.

Patients with lung cancer with EGFR or ALK mutations can be treated with targeted therapy against these mutations. The effects of these targeted therapies can first be examined in patient-derived organoids to predict responses to therapy and control for the presence of additional genetic abnormalities that render these targeted therapies ineffective due redundancies or downstream mutations in the targeted pathways. Recently, immune checkpoint inhibitors that block PD-1 checkpoint such as nivolumab and pembrolizumab or block PD-L1 such as atezolizumab were approved for treatment of non-small cell lung cancer after first receiving chemotherapy. Patients with highest levels of PD-L1 (˜30% of patients) have higher chances of response. Organoids could be used to examine responses to checkpoint inhibitors by examining cytokine release and lymphocyte activation upon coculture of organoids with patient derived lymphocytes such as those separated from tumor infiltrating lymphocytes (TILs) or in the immune humanized mice engrafted with patient derived organoids.

In another aspect of this method, clonally targeted therapies can be determined by testing the effect of a therapeutic agent on multiple organoids derived from subsequently determined dominant clones of lung cancer cells identified in the tumor tissue from a patient, and comparing to the effect of the therapeutic agent on organoids derived from normal cells of the same patient.

In another aspect, the invention provides a cell culture (e.g., organoid) medium supplemented with FBS, Insulin and bFGF. In another embodiment, the invention provides a cell culture (e.g., organoid) medium supplemented with FBS, Insulin, bFGF, EGF, hydrocortisone, Cholera Toxin, Transferrin, and Sodium Selenite. In another embodiment, the invention provides a cell culture (e.g., organoid) medium supplemented with FBS, Insulin, bFGF, EGF, hydrocortisone, Cholera Toxin, Transferrin, Sodium Selenite, Penicillin and Streptomycin. In a preferred embodiment, the medium is a commercially available cell growth medium such as ADMEM (ThermoFisher scientific).

In another aspect, the invention provides kits to make an organoid from a single cell. In an embodiment, a kit contains containers for a differentiation medium and an organoid medium as previously described. The containers may also contain the necessary supplements (growth factors, antibiotics, hormones, vitamins, amino acids, and combinations thereof) for a differentiation medium and an organoid medium. The kit may further include the necessary components for a 3-dimensional culture device, for example, plates, and/or materials for an extracellular matrix, e.g. Matrigel™. The kit may further contain a set of instructions to perform the methods of making an organoid from a single cell as previously described.

In another embodiment, the present invention provides a mouse with an implanted patient-specific lung organoid. In one embodiment, the mouse is a humanized mouse. In another embodiment, the mouse is a human immune system (HIS)-reconstituted mouse. In another embodiment, the mouse is non-obese diabetic (NOD)-Rag (−)-γ chain (−) (NRG) mouse. In another embodiment, the mouse is a RAG1/2 or an NSG immune-deficient PDX mouse.

Methods of making HIS-reconstituted mice are known in the art and disclosed for example by Drake et al. (2012) Cell Mol Immunol 9:215-24 and Harris et al. (2013) Clinical and Experimental Immunology 174:402-413. In accordance with one aspect of the present invention, human stem cells from patient, for example from a diagnostic bone marrow or blood sample or HLA-matched, are transplanted into neonatal NRG mice to engraft components of the patient's immune system. Methods of making NSG immune-deficient PDX mice are also known in the art and disclosed for example by Zhang et. al., (2015) Anticancer Res 35:3755-3759. The mice are later subjected to grafting with lung organoids derived from lung cells of the same patient orthotopically in the mouse left lung. The mice are useful for identifying new treatments, assessing responses to therapy, and evaluating combination therapies.

The following non-limiting examples serve to further illustrate the invention.

### Example 1

In the experiments described below, organoids from lung adenocarcinoma tissue, the most common subtype, were generated. Working conditions for lung organoids were established. Lung-specific signaling and lung specific expression analysis of different cell lineages present in normal and tumorigenic lungs were examined. Lung organoids were propagated in NSG immune deficient mice to generate humanized PDX mice with lung patient-derived organoids (PDOs). Table 2 below includes the media and culture conditions in a typical embodiment of producing lung tissue organoids.

TABLE 2
Lung Organoid Media

| Primary | | Process | 2D Culture | 3D Culture | Days to |
|---|---|---|---|---|---|
| Tissue | Collection Media | Time | (phase I) | (Phase II in Matrigel) | organoids |
| Lung | RPMI medium + 10% | Dissociate | ADMEM medium + 5% | ADMEM medium + 5% | Phase I: 7-10 |
| | FBS Penicillin (3,000 | tissue for | FBS + Penicillin (1,000 | FBS + Insulin | Phase II: 28 |
| | Units/mL) + Streptomycin | 2-12 hours | Units/mL) + Streptomycin | (50 mg/mL) + bFGF | |
| | (300 μg/mL) | | (100 μg/mL) | (10 mg/mL) + EGF | |
| | | | | 20 mg/mL + Hydrocortisone | |
| | | | | 1 mM + Cholera Toxin | |
| | | | | (10 ng/mL) + Transferrin | |
| | | | | (5.5 ng/mL) + Sodium | |
| | | | | Selenite | |
| | | | | (7 ng/mL) + Penicillin | |
| | | | | (1,000 | |
| | | | | Units/mL) + Streptomycin | |
| | | | | (100 μg/mL) | |

### Example 2

The generation of PDOs from normal and diseased lung biopsies, with organoids containing stem and progenitor cells and differentiated pulmonary epithelial cell types, allows for the generation of not only personalized therapy for lung cancer, but also better models for other human lung diseases. To generate lung PDOs, the optimum growth conditions to produce the highest organoid forming efficiency (OFE) were first determined. These conditions were then utilized to make organoids from primary human lung normal and cancer tissues. Under an IRB-approved protocol, specimens from high-risk lung cancer cases were collected and processed within 15 minutes of surgery. Areas containing tumor as deemed by the pathologist and normal adjacent tissue counterpart were microdissected. A 3D culture system fit for growth of lung cells was first developed by isolating epithelial cells microdissected from primary lung cancer specimens. Qualified pathologists confirmed their lung origin from the corresponding H&E and molecular assays. Cells were placed in 3D droplet culture chambers containing Matrigel, to mimic the basal lamina of the normal lung tissue, and growth factors in conditions that permit cellular self-organization of organoid forming cells. Lung cells were embedded as single cells in 3D-well plates. Organoid formation was then followed microscopically daily for 2-4 weeks. Whether the 3D culture conditions are optimized for maintenance of expression of the lung lineage-specific markers and their functional secretory profile was examined. Next, these media were tested directly on primary lung cells from tumor and normal adjacent tissue (NAT) from lung resected tissues from multiple patients, and it was observed that the OFE was superior when cells were grown in ADMEM rather than any other lung tissue growth media. Moreover, the addition of 5% FBS was required to maintain both 2D and 3D cultures to generate lung tissue specific organoids. Pathological examination has always confirmed that the tissues dissociated were from lung cancer foci (>90% tumor), suggesting that normal cell overgrowth is improbable. Nevertheless, to exclude this possibility, single epithelial cells were isolated from both NAT and cancer tissues and their respective OFE were evaluated. The OFE of cancer foci-derived cells was significantly higher compared to that of normal tissue-derived cells. Next, the ability to dissociate primary 3D organoids and generate serial passages of organoids from lung organoid derived single cells was evaluated. These experimental procedures ensured that these lung organoids are derived from single cells, indicating that they are generated from undifferentiated or multipotent stem cells. When dissociated at the single cell level and replated, the organoid-forming cells showed serial clonogenic capacity and formed similar numbers of organoids in secondary plating, suggesting the maintenance of expression of stemness factors in these culture conditions. Thus, these lung organoids harbor stem cell characteristics: self-renewal capability for sustaining tumor propagation and progression, while retaining the capacity to differentiate into offspring(s) resembling the main cellular populations of the lung tissue. It was speculated that lung cancer organoids stemmed from a single ancestor cell endowed with stem-like traits that progressively gives rise to a differentiated and more specialized progeny comprising all the main lung lineages. To further investigate this assumption, experiments were first conducted at the labeled single cell level. Primary lung cancer cells were lentivirally engineered to express enhanced green fluorescent protein (EGFP) and subsequently were embedded as single labelled cells in 3D-well plates. Organoid formation was then followed microscopically daily for three full weeks. It was observed that the clear majority of the resulting organoids expressed EGFP suggesting their single cell origin.

Next, whether the addition of 5% FBS was required to maintain both 2D and 3D cultures to generate lung tissue specific organoids was examined. Based on OFE in 3D cultures, FBS was found to increase OFE when 10⁵ cells are plated, but is dispensable, since organoids are still formed. However, the addition of FBS was found to be indispensable when starting with lower cell density at 10³ cells or less.

In order to further demonstrate that the clonally proficient single cell derived organoids are able to generate differentiated lung cells, and to confirm that the organoids are derived from the lung tissue and have specific expression of lung specific markers, RNA was extracted from the normal and lung cancer tissues and their corresponding organoids, and gene expression profiling was performed for lung specific markers, previously identified to be critical for lung tissue development and lung cell lineage determination.

Mechanisms that regulate the development of lung tissues, lung cell lineage specification, and subsequent progenitor patterning and growth have been described. The onset of lung specification within the endoderm is accompanied by the expression of Nkx2.1, the earliest and most specific marker of lung endoderm. The NKx2.1+ cells in this region of the embryonic lung are airway progenitor cells that give rise to the mature airway E-Cadherin expressing epithelial cells in the trachea, bronchus, and bronchioles. In contrast, BMI1, SOX9, FOXA2, and ID2 are expressed in the distal embryonic lung bud tip and mark both multipotent embryonic lung progenitor population and bronchioalveolar adult distal lung epithelial stem cells that are capable of producing all of the cell types of the airway and alveoli cells. Bronchioalveolar differentiated lung cells express the bronchiolar club cell (Clara) marker, CCSP, and the alveolar type 2 cell marker, SPTPC. The organoids were evaluated for markers of lung stem and differentiated cells, including assessing the lung epithelium lineage-specific markers. The lung tissue origin of organoids was confirmed by detecting the overexpression of NKX2.1 in lung cancer PDOs.

In 3D organoids from primary lung cancer tissue 042, enhanced NKX2.1 expression was detected in 3D organoids and subsequently expanded organoids in 3D culture. Organoids persisted in culture for over 50 days so far and still ongoing, and developed well-organized airway epithelial structures that included cell types found in the lung epithelium, including basal and ciliated cells.

3D organoids from primary lung cancer tissue 042 demonstrated higher expression of bronchioalveolar adult distal lung epithelial stem cell markers BMI1, SOX9 and FOXA2, compared to 2D cultured cells. These organoids also expressed much less of the basal cell marker P63 in both 2D and 3D culture conditions, compared to normal cells, as expected for lung cancer tissues. Organoids also possessed terminal differentiation markers such as the distal alveolar type 2 cell marker SPTPC. The 3D organoids from primary lung cancer tissue 042 had significantly higher expression of the terminal differentiation markers for multi-ciliated cells FOXJ1 and alveolar type 1 cell marker HopX. These data suggest that PDOs were generated from lung adenocarcinoma, and that organoids have cellular expression of progenitor and terminally differentiated lung cell subtypes.

### Example 3

African American (AA) men have an 18% higher incidence and 20% higher mortality rate from lung cancer than European American (EA) men. Notably, these disparities remain after controlling for smoking or social determinants of health. Prior studies have focused on descriptive associations with disparities. The organoids described herein can be used to uncover the key molecular mechanisms driving non-small cell lung cancer (NSCLC) racial disparities while addressing the urgent need to develop effective therapeutic strategies that could soon improve survival in minority patients.

It was reported that elevated IL6 (which activates JAK/STAT3) is more strongly associated with increased lung cancer risk among AAs than European Americans (EAs). Previous reports show that hallmarks of JAK/STAT3 activity are enriched in NSCLCs from AAs vs EAs, such as stem cell/invasion pathways. It has also been shown that: 1) NSCLCs from AAs have a significantly higher mutational burden in the STAT3 phosphatases PTPRD and PTPRT (27.7% vs 16.1% in AAs vs EAs); 2) Mutations in PTPRD and PTPRT result in aberrant STAT3 activation; 3) JAK/STAT inhibitors have significantly more potent antitumor activities in NSCLC from AAs vs EAs; and 4) the STAT3 inhibitor, BBI608, had effective anti-tumor activity against PTPRT-mutant NSCLC in vivo. From these results, we surmise that AA NSCLCs are more addicted to JAK/STAT3 signaling as compared to EAs, and the organoids described herein can be used to uncover if STAT3 blockade has potent anti-tumor activity in NSCLC with PTPRD/T loss-of-function mutations, thus providing treatments that could impact AA NSCLC patients more and hence reduce lung cancer disparities.

Use of NSCLC-derived 3D organoids from AA patients will provide a more accurate representation of the impact of race-associated JAK/STAT3 pathway mutations. We surmise that mutations in STAT3 phosphatases PTPRD/T cause aberrant STAT3 activation and drive the tumorigenic process in NSCLC, and their higher prevalence in AAs leads to cancer health disparities. The organoids described herein can be used to uncover the mechanisms by which PTPRD/T mutations control STAT3 activity in molecularly defined AA NSCLC organoids from primary and patient-derived xenograft (PDX) models.

Organoids from primary (cells) and PDXs of AA NSCLC and from primary (cells) and PDXs of EAs were generated according to the methods of Examples 1-3. The 3D cultured organoids matched (vis a vis H&E histology and expression of lung-specific markers) the patient's primary NSCLC tumor from which the organoids were derived.

The organoids described herein can be used to examine the structural and functional effects of PTPRD/T mutations in a) STAT3 activation and phosphatase modeling; b) cell transformation in anchorage independent growth; and c) sensitivity to STAT3 blockade in NSCLC cells and 3D organoids from primary and PDXs of AA NSCLC with CRISPR-mediated PTPRD/T knockout and rescue studies. Use of CRISPR/Cas9, NSCLC-derived 3D organoids, and mutant PTPRD/T-featuring PDX from AA patients will provide a more accurate representation of the impact of race-associated JAK/STAT3 pathway mutants.

To test if predicted loss-of-function mutations in PTPRD/T result in elevated p-STAT3 in NSCLC tissues, we analyzed TCPA reverse-phase protein array (RPPA) and TCGA sequencing data. There was a significant association between mutations and increased p-STAT3. We transfected immortalized human bronchial epithelial (Beas2b) cells (pSTAT3-low) with WT or mutant PTPRD (R1692L) or PTPRT (R1335F). Both mutations promoted upregulation of pSTAT3.

To test if PTPRD/T mutations contribute to IL6-mediated STAT3 activation in a race-associated manner, the organoids described herein can be treated with recombinant human IL6 (50 ng/ml) or control antibody in serum-free conditions at various time-points (1, 3, 6 and 24 hrs), and assessed for STAT3 activation by measuring pSTAT3 (normalized to total STAT3) and expression of STAT3 targets (e.g. Survivin, CycinD1, MYC, etc). For testing PTPRD/T activation of STAT3, the organoids described herein can be used to silence the mutant gene in NSCLC cell lines first using a lenti shRNA approach targeting the 5′UTR, and reintroduce the mutant (for rescue) and WT gene at a low MOI against scramble shRNA and empty vector controls.

Because PTPRD/T mutations increase STAT3 activity, it stands to reason that tumors addicted to activated STAT3 via PTPRD/T mutations would be sensitive to STAT3 inhibition. The organoids described herein can be treated with: a) STAT3 inhibitor BBI608; b) standard NSCLC chemotherapy (carboplatin plus paclitaxel); or c) BBI608 plus chemotherapy. Organoid bioluminescence (BLI), cell viability measured by intracellular ATP, cell proliferation (Ki67), survival [Necrosis by calcein permeability or autophagy by Cyto-ID kit (Enzo)], pSTAT3 levels, 3D-migration and invasion into extracellular matrix assays for functional studies can be assessed. To assess the effects of mutant PTPRD/T on in vivo tumor growth using NSG subcutaneous (SC) xenografts, the organoids described herein can be used to assess metastatic behavior by injecting BLI tumor cells into the right ventricle of NSG mice and imaging reduced metastases via IVIS, following established procedures.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein by reference in their entireties.

We claim:

1. A method of making an organoid from a mammalian lung tissue in vitro comprising: isolating cells from a mammalian lung tissue to provide isolated cells; culturing the isolated cells in a differentiation medium for a time sufficient to enrich for stem cells and induce differentiation; and amplifying one or more of the cells by culturing in an extracellular matrix in an organoid medium for a time sufficient to produce organoids that exhibit endogenous three-dimensional organ architecture.

2. The method of claim 1 wherein the differentiation medium comprises fetal bovine serum (FBS).

3. The method of claim 1 wherein the organoid medium comprises FBS, Insulin and basic fibroblast growth factor (bFGF).

4. The method of claim 3 wherein the organoid medium further comprises one or more of epidermal growth factor (EGF), hydrocortisone, Cholera Toxin, Transferrin, and Sodium Selenite.

5. The method of claim 1 wherein the mammalian tissue is a human tissue.

6. The method of claim 5 wherein the human tissue is human lung tissue.

7. The method of claim 6 wherein the human lung tissue is primary human normal lung tissue, or primary human lung cancer tissue.

8. The method of claim 1 wherein the organoids comprise epithelial cells.

9. The method of claim 1 wherein the time sufficient to produce organoids is about twenty-eight days.

10. The method of claim 3 wherein the FBS is present at a concentration of about 1-10%, the Insulin is present at a concentration of about 1-100 mg/mL, and the bFGF is present at a concentration of about 1-50 mg/mL.

11. The method of claim 3 wherein the FBS is present at a concentration of about 5%, the Insulin is present at a concentration of about 50 mg/mL, and the bFGF is present at a concentration of about 10 mg/mL.

12. The method of claim 4 wherein the medium comprises EGF at a concentration of about 1-50 mg/mL, hydrocortisone at a concentration of about 0.1-10 mM, Cholera Toxin at a concentration of about 0.1-100 ng/mL, Transferrin at a concentration of about 0.5-25 ng/mL, and Sodium Selenite at a concentration of about 0.5-25 ng/mL.

13. The method of claim 1 wherein the isolated cells are sorted for the presence of at least one marker selected from the group consisting of NKx2.1, CCSP, surfactant protein precursor C (SPTPC), FOXJ1 and HopX.

14. A lung organoid comprising epithelial cells, the organoid exhibiting endogenous three-dimensional organ architecture.

15. A lung organoid derived in vitro from primary lung normal tissue, wherein the organoid comprises epithelial cells and exhibits endogenous three-dimensional organ architecture.

16. A lung organoid derived in vitro from primary lung cancer tissue, wherein the organoid comprises epithelial cells and exhibits endogenous three-dimensional organ architecture.

17. A cell culture medium supplemented with FBS, Insulin and bFGF.

18. A cell culture medium supplemented with FBS, Insulin, bFGF, EGF, hydrocortisone, Cholera Toxin, Transferrin and Sodium Selenite.

19. The cell culture medium of claim 17 further comprising EGF and hydrocortisone.

20. A kit comprising the cell culture medium of claim 19.

21. A method for identifying an agent having anticancer activity against lung cancer cells from a patient comprising selecting at least one test agent, contacting a plurality of lung organoids derived from lung cancer cells from the patient with the test agent, determining the number of lung organoids in the presence of the test agent and the absence of the test agent, and identifying an agent having anticancer activity if the number or growth of the organoids derived from lung cancer cells from the patient is less in the presence of the agent than in the absence of the agent.

22. A method of personalized treatment for lung cancer in a subject comprising: selecting at least one form of treatment, contacting a plurality of lung organoids comprising with the form of treatment, wherein the organoids are derived from lung cancer cells from the subject, determining the number of lung organoids in the presence of the treatment and the absence of the treatment, and selecting the treatment if the number or growth of the lung organoids is less in the presence of the treatment than in the absence of the treatment.

23. The method of claim 22 further comprising treating the subject with the selected treatment.

24. A method of personalized treatment for lung disorders in a subject comprising: selecting normal lung cells to generate organoids, wherein the organoids are derived from lung normal cells from the subject, or HLA-matched donors, generating normal patient-specific or HLA-matched lung organoids, and using such organoids for personalized therapies for lung disorders.

25. A humanized mouse engrafted with components of a patient's immune system and comprising a lung organoid derived from the patient's lung cell grafted into the mouse.

26. The method of claim 21, further comprising providing a mouse engrafted with lung cancer cells from the patient and containing a tumor formed from the lung cancer cells; administering the identified agent having anticancer activity to the mouse; and determining if the tumor size is reduced in the presence of the identified agent.

27. The method of claim 21, further comprising providing a humanized mouse engrafted with components of a patient's immune system and lung cancer cells from the patient and containing a tumor formed from the lung cancer cells; administering the identified agent to the humanized mouse; and comparing the size of the tumor in the humanized mouse with components of a patient's immune system to the size of the tumor in the mouse in which the identified agent was administered; and determining if the size of the tumor in the humanized mouse with components of a patient's immune system is reduced relative to the size of the tumor in the mouse in which the identified agent was administered.

28. The method of claim 21 or 27, further comprising providing a humanized mouse engrafted with lung cancer cells from the patient and containing a tumor formed from the lung cancer cells; administering a control agent to the humanized mouse engrafted with lung cancer cells from the patient; and comparing the size of the tumor in the humanized mouse engrafted with lung cancer cells from the patient to the size of the tumor in the mouse in which the identified agent was administered; and determining if the size of the tumor in the mouse in which the identified agent was administered is reduced relative to the size of the tumor in the humanized mouse engrafted with lung cancer cells from the patient.

29. The method of claim 21, wherein the patient is an African American (AA), and the at least one test agent is an inhibitor of JAK/STAT3 activity.

30. The method of claims 22-24, wherein the subject is an AA, and the form of treatment is an inhibitor of JAK/STAT3 activity.

31. The method of any one of claims 21-24 and 26-30, wherein the organoids exhibit endogenous three-dimensional organ architecture.

32. The lung organoid of any of claims 14-16, wherein the lung organoid is derived in vitro from primary lung tissue from an AA.
